(19) United States
(12) Patent Application Publication
(10) Pub. No.: US 2021/0298625 A1
(43) Pub. Date: Sep. 30, 2021

(54) SYSTEM AND METHOD FOR DETECTING AND PREDICTING AN OCCURRENCE OF CARDIAC EVENTS FROM ELECTROCARDIOGRAMS

(71) Applicant: COHERE-MED INC., Dover, DE (US)

(72) Inventors: Srikanth MUTHYA, Bangalore (IN); Renjith Somanathan NAIR, Bangalore (IN); Krishna Prasad VELUTHAKKAL, Palakkad (IN); Anusha PRAKASH, Bangalore (IN)

(73) Assignee: COHERE-MED INC., Dover, DE (US)

(21) Appl. No.: 16/829,978

(22) Filed: Mar. 25, 2020

(51) Int. Cl. A61B 5/04; A61B 5/00; A61B 5/0472; A61B 5/0456; G16H 10/60

(52) U.S. Cl. CPC A61B 5/04012; A61B 5/7275; A61B 5/7267; G16H 10/60; A61B 5/0472; A61B 5/0456; A61B 5/0022

(57) ABSTRACT

A method for training a system for predicting a probability of occurrence of cardiac event is provided. The method includes obtaining sets of electrocardiograms of subjects, each set of electrocardiograms comprising at least one electrocardiogram each obtained, a) prior to and b) during or after an occurrence of a cardiac event, or both. The method includes extracting a first approximation of a time series data. The method also includes obtaining electrocardiograph signals that produced the electrocardiograms. The method also includes creating a training dataset from the extracted time series signals and the electrocardiograph signals for training a first and a second approximation of an electrocardiogram for representing an electrocardiograph signal that produced the electrocardiogram. The method further includes training a second model for extracting a cardiac marker from the second approximation of the electrocardiogram of the subject and calculating a probability of occurrence of a cardiac event.

### FIELD

The present disclosure generally relates to prediction of an occurrence of a cardiac event and more particularly to techniques relating to machine learning systems for detecting a prior occurrence of a cardiac event or predicting an occurrence of a cardiac event or both from an image of an electrocardiogram (ECG).

### BACKGROUND

Electrocardiography, commonly referred to as ECG, continues to be broadly applicable in diagnosis of heart disease. Growth in cardiac diseases has led to a need for accurate diagnoses based on an interpretation of ECGs from a variety of systems, across a diverse population. However, the accurate recording and interpretation of the ECG is critical.

Considering the fact that it is humanly impossible to interpret every ECG, computerized interpretation of ECGs using algorithms has gained importance in recent years. The algorithms may be heuristic (experience-based rules that are deterministic) or statistical (probabilistic) in structure. Heuristic algorithms were originally designed to incorporate discrete measurement thresholds into a decision tree or Boolean combinations of criteria. Statistical algorithms circumvent problems of diagnostic instability that are associated with small serial changes around discrete partitions by adding a probability statement to the diagnosis. These may be based on Bayesian logic.

Other statistical methods use discriminant function analysis, which can use continuous ECG parameters in addition to discrete variables to produce a point score. These algorithms tend to be more reproducible than earlier heuristic methods, even though they still may result in discrete thresholds for diagnostic statements. Neural networks differ from conventional discriminant function analysis in the way they are trained, in the resulting classifier, and in their derived decision boundaries.

Furthermore, statistical methods depend on a database of well documented cases to find the optimal ECG parameters for use. Such a database must be large enough for the results to be statistically reliable. In addition, the database must contain enough cases with varying degrees of abnormality, ranging from mild to severe cases, and a representative distribution of common compounding conditions. The statistics of well documented populations has been used to develop diagnostic algorithms that no longer simply mimic the human reader.

In recent years, another diagnostic approach in cardiac diseases such as two-dimensional echocardiography has become the favored reference standard but is now being challenged by three-dimensional echocardiography, computerized tomography, and magnetic resonance imaging. Although these newer imaging techniques provide a more accurate assessment of ventricular myocardial mass than does the ECG, they do not obviate the clinical use of the ECG. The greater convenience and lower cost of the ECG continue to support its widespread use for the diagnosis of ventricular disorders in clinical practice, epidemiological studies, and clinical trials. In addition, some ECG abnormalities have been shown to have independent clinical prognostic value. The evolution of these new diagnostic approaches provides a compelling reason to reassess the role of the ECG in detecting cardiac disorders and related abnormalities.

The situation regarding precise identification of the state of the heart by characterizing the ECG signals gets even more varied, with different measurement systems available that may have different technical specifications that result in significant differences in the measurement of amplitudes, intervals and diagnostic statements. A misclassification can definitely result in increased cost and burden for the individual. Moreover, there are too few expert cardiologists and a general practitioner may have an electrocardiograph but is not capable of reading the electrocardiogram. Even experts may not be able to recognize the markers in an electrocardiogram. Several commercially available products today implement heuristic algorithms using time series data or application of pattern recognition on images for detecting a cardiac event. However, these are limited to continuous learning of newer patterns, learning from misclassification and are subject to large variations in interpretations. Moreover, the database against which patterns are compared are very small.

### SUMMARY

This summary is provided to introduce a selection of concepts in simple manners that are further described in the detailed description of the disclosure. This summary is not intended to identify key or essential inventive concepts of the subject matter nor is it intended to determine the scope of the disclosure.

To overcome one or more of the above mentioned problems, a system configured for and a method of predicting an occurrence of a cardiac event from an electrocardiogram using data transformation and cross-learning techniques is needed. Preferably predictions by such a system and method have a greater accuracy and recall scores than available hitherto. Moreover, a machine learning system for extracting a cardiac marker for quantifying the risk factor for a subject is needed. Such predictions are meant to be used for managing the subject proactively.

In the present disclosure, the term electrocardiogram is used to refer to the strip of paper on which electrical signals associated with the functioning of the heart are recorded as a two-dimensional graph, as is well known in the field. Similarly, the term electrocardiograph is used to refer to the device, along with the electrical leads, that is used to produce electrocardiograms. In recent times electrocardiographs that store electrocardiograms in a portable document file, in the .pdf format, have become available. All such variants are included under the term electrocardiogram.

Briefly, according to an exemplary embodiment of this disclosure, a system for predicating and detecting an occurrence of a cardiac event from an electrocardiogram of a subject under test is provided. The system includes a processor with a memory. The memory storing a plurality of modules configured for at least one of detecting and predicting an occurrence of a cardiac event from the electrocardiogram of the subject under test. The plurality of modules are characterized by a first module having been trained using machine learning and configured for: obtaining a plurality of sets of electrocardiograms of a plurality of subjects, wherein each set of electrocardiograms of each subject comprises at least one electrocardiogram obtained prior to an occurrence of a cardiac event and at least one of an electrocardiogram obtained during an occurrence of the cardiac event and an electrocardiogram obtained after an occurrence of a cardiac event; extracting a first approximation of a time series signal from each of the electrocardiograms; obtaining each of an electrocardiograph signal that produced each of the electrocardiograms; wherein the electrocardiograph signal is a raw time series signal; creating a training dataset from the extracted time series signals and the electrocardiograph signals and training a first model on the training dataset for extracting a second approximation of a time series signal from the electrocardiogram of the subject under test, wherein the second approximation represents an electrocardiograph signal that produced the electrocardiogram of the subject under test. The plurality of modules are characterized by a second module having been trained using machine learning, on the training dataset for extracting a cardiac marker from the second approximation of the time series signal extracted from the electrocardiogram of the subject under test and calculating a probability of occurrence of a cardiac event.

Briefly, according to an exemplary embodiment of this disclosure, a method for training one or more models, using machine learning, for predicting a probability of occurrence of cardiac event from an electrocardiogram of a subject under test is provided. The method includes obtaining a plurality of sets of electrocardiograms of a plurality of subjects, wherein each set of electrocardiograms of each subject comprises at least one electrocardiogram obtained prior to an occurrence of a cardiac event and at least one of an electrocardiogram obtained during an occurrence of the cardiac event and an electrocardiogram obtained after an occurrence of a cardiac event. The method includes extracting a first approximation of a time series signal from each of the electrocardiograms. The method also includes obtaining each of an electrocardiograph signal that produced each of the electrocardiograms; wherein the electrocardiograph signal is a raw time series signal. The method also includes creating a training dataset from the extracted time series signals and the electrocardiograph signals and training a first model on the training dataset for extracting a second approximation of a time series signal from the electrocardiogram of the subject under test; wherein the second approximation represents an electrocardiograph signal that produced the electrocardiogram of the subject under test. The method further includes training a second model on the training dataset for extracting a cardiac marker from the second approximation of the time series signal extracted from the electrocardiogram of the subject under test and calculating a probability of occurrence of a cardiac event.

The summary above is illustrative only and is not intended to be in any way limiting. Further aspects, exemplary embodiments, and features will become apparent by reference to the drawings and the following detailed description.

### BRIEF DESCRIPTION OF THE FIGURES

These and other features, aspects, and advantages of the exemplary embodiments can be better understood when the following detailed description is read with reference to the accompanying drawings in which like characters represent like parts throughout the drawings, wherein:

FIG. 1 illustrates an exemplary environment of a system for training one or more models, using machine learning, for predicting a probability of occurrence of cardiac event from an electrocardiogram (ECG) of a subject under test, in accordance with an embodiment of the present disclosure;

FIG. 1-A is an exemplary illustration of input data which is a time series signal also referred as the electrocardiograph signal that produced each of the ECG from the sets of ECGs of a plurality of subjects; in accordance with an embodiment of the present disclosure;

FIG. 1-B is an exemplary illustration of input data which is a combination of images obtained from the sets of ECGs of a plurality of subjects, in accordance with an embodiment of the present disclosure;

FIG. 2-A-D is an exemplary illustration of a peak detection algorithm implemented for extracting time series signals from each of the sets of the electrocardiograms, in accordance with an embodiment of the present disclosure;

FIG. 2-E is an exemplary illustration of a method for predicting and detecting an occurrence of a cardiac event, by performing image analysis, on an electrocardiogram computed from near real time electrocardiograph signals of a subject under test, in accordance with an embodiment of the present disclosure;

FIG. 3 illustrates an exemplary environment for detecting a previous occurrence of cardiac event from an electrocardiogram of the subject under test, in accordance with an embodiment of the present disclosure;

FIG. 3-A is an exemplary illustration showing phases of analysis on the input data which is an ECG strip, for detection of cardiac event in accordance with an embodiment of the present disclosure;

FIG. 4 is a flow chart illustrating a method for estimating at least one of a presence of a cardiac marker indicating a prior occurrence of a cardiac event and calculating a probability of a future occurrence of a cardiac event from an electrocardiogram of a subject under test, in accordance with an embodiment of the present disclosure; and

FIG. 5 illustrates a block diagram of an electronic device implemented according to an embodiment of the present disclosure.

Further, skilled artisans will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION

For the purpose of promoting an understanding of the principles of the invention, reference will now be made to the embodiments illustrated in the figures and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not comprise only those steps but may comprise other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skill in the art to which this invention belongs. The system, methods, and examples provided herein are illustrative only and not intended to be limiting.

In addition to the illustrative aspects, exemplary embodiments, and features described above, further aspects, exemplary embodiments of the present disclosure will become apparent by reference to the drawings and the following detailed description.

Embodiments of the present disclosure disclose a system and a method for predicting an occurrence of a cardiac event from an electrocardiogram of a subject under test, using data transformation and cross-learning techniques. The method and system are oriented towards predicting an occurrence of a cardiac event accurately, precisely and with a high recall scores. Further, embodiments of the present disclosure particularly disclose a system and a method configured for training one or more models, using machine learning, for predicting a probability of occurrence of a cardiac event from the electrocardiogram of the subject under test. The present disclosure discloses implementing data transformation techniques for converting discrete raw values obtained from the electrocardiograph systems to electrocardiogram (ECG) images (as seen by humans), and vice versa, to predict and characterize heart rhythm conditions of the subject under test, using neural networks, computer vision techniques validated across a large dataset of acquired ECG from longer encounters of subjects in critical care, Holter of ambulatory patients and event monitoring networks. Thus, the system disclosed herein is configured for predicting the occurrence of cardiac adverse conditions of the subject under test, ahead of time, to help manage the subject better for improving clinical outcomes.

In the description of some embodiments, the words ‘subject’, ‘patient’ and ‘individual’ may have the same meaning and may have been used interchangeably. Furthermore, the term ‘electrocardiogram’ or ECG′ used in the description refer to a plot showing the electrical activity of a human heart, on a strip chart. The term ‘electrocardiograph’ used in the description refers to the device and the system of electrodes detecting the electrical activity of the heart and the detected signal (also referred electrocardiograph signal) used to produce an electrocardiogram. The term extracted time series signal (first approximation) used in the description refers to the data resulting from the analysis of sets of electrocardiograms of a plurality of subjects. Further, the term ‘raw time series signal’ used in the description refers to the signal detected by the electrocardiograph and obtained in real time. It is also referred to as the electrocardiograph signal. In both cases, the signal is a digital representation of the analog signals detected by the electrocardiogram. Embodiments of the present invention will be described below in detail with reference to the accompanying figures.

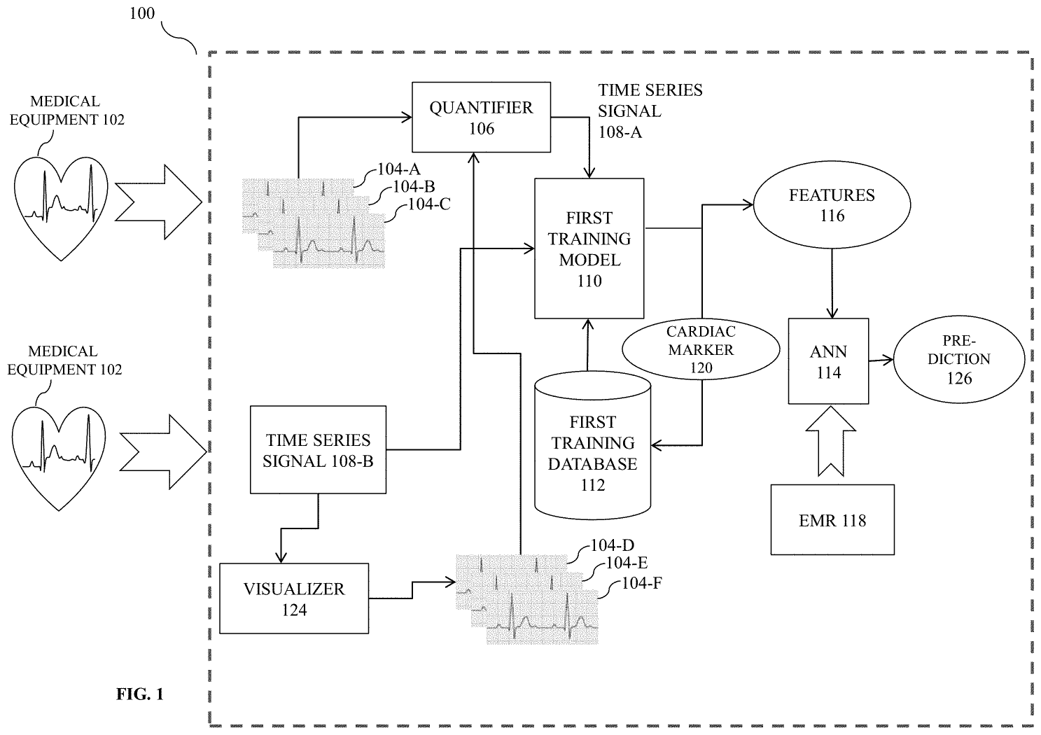

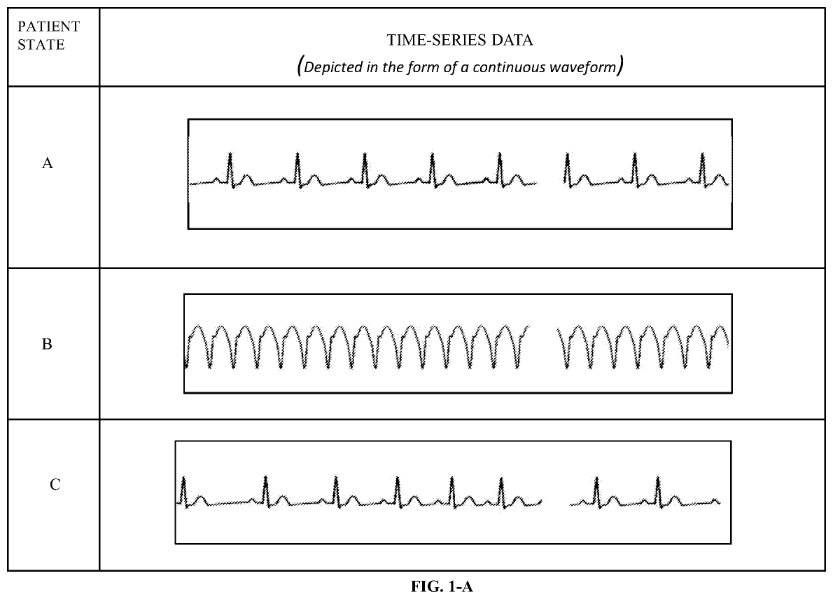

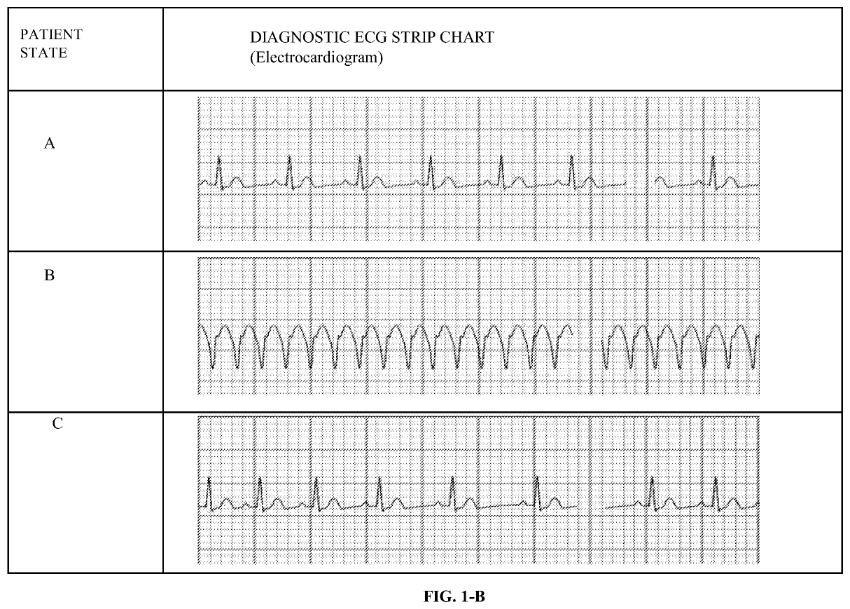

FIG. 1 illustrates an exemplary environment of a system 100 for training one or more models, using machine learning, for predicting a probability of occurrence of cardiac event from an electrocardiogram of a subject under test, in accordance with an embodiment of the present disclosure. FIG. 1 illustrates, a medical equipment such as electrocardiograph 102, a plurality of sets of electrocardiograms (ECG 104-A through 104-C) of the plurality of subjects, a quantifier 106, a time series signals 108-A extracted by the quantifier 106, an electrocardiograph signal, that is, the raw time series signal 108-B, a first convolutional neural networks based training model 110, a first training database 112, a second artificial neural networks based training model 114, one or more features 116, an electronic medical record 118, a cardiac marker 120 and a predicted signal 126. In addition, FIG. 1 illustrates a visualizer 124 and sets of electrocardiograms (ECG 104-D through 104-F). Each block is explained in detail further below. Furthermore, the manner in which the models are trained, using machine learning and used for estimating at least one of a presence of the cardiac marker 120 indicating a prior occurrence of a cardiac event and calculating a probability of a occurrence of a cardiac event from an electrocardiogram (image strips) of the subject under test, is described in detail further below.

It is to be noted that the system 100 is configured for predicting an occurrence of a cardiac event from the electrocardiogram of the subject under test. The system 100 includes a processor with a memory (not shown in FIG. 1). The memory is configured for storing a plurality of modules (not shown) configured for predicting the occurrence of a cardiac event, from the electrocardiogram of the subject under test. A first module having been trained using machine learning for training a first training model 110 on a training dataset for extracting a second approximation of a time series signal from an electrocardiogram of the subject under test. A second module having been trained using machine learning for training a second training model 114, on the training dataset, for extracting a cardiac marker from the second approximation of the time series signal extracted from the electrocardiogram of the subject under test and calculating a probability of occurrence of a cardiac event.

The models (the first training model 110 and the second training model 114) are trained using an input data which is a combination of images obtained from the sets of ECG (104-A-C) of the plurality of subjects and the electrocardiograph signal 108-B that produced each of the ECG (104-A-C). In one example, the ECG (104-A-C) illustrates, a plot showing the electrical activity of a heart of the plurality of subjects on a strip chart. In another example, the electrocardiograph signal 108-B is the detected signal (raw time series signal) obtained from the electrocardiograph 102. The electrocardiograph 102 is a medical equipment, comprising electrodes for detecting the electrical activity of the heart and the detected signal is used to produce the electrocardiogram (104-A-C). In the same example, the electrocardiograph signal 108-B is the raw time series signal obtained in real time and that produced each of the electrocardiograms (104-A-C) of the plurality of subjects.

Referring to FIG. 1, a plurality of sets of electrocardiograms (104-A-C) of the plurality of subjects is received as the first part of the input data. Each set of ECGs comprises at least one ECG (104-A) obtained prior to the occurrence of a cardiac event (state A), at least one of ECG (104-B) obtained during an occurrence of a cardiac event (state B) and an ECG (104-C) obtained after the occurrence of a cardiac event (state C). FIG. 1-B is an exemplary illustration of the input data which is a combination of images obtained from the sets of ECGs (104-A-C) of the subjects. FIG. 1-A is an exemplary illustration of the time series signal 108-B that produced each of the ECGs (104-A-C). The ECG data collected and received across different states (state A, state B, and state C) of the subject over a period of time is as shown in FIG. 1-A-B. For example, state A would be prior (pre-condition) to the occurrence of a cardiac event, state B (at-condition) would be at the time of or during the occurrence of a cardiac event and state C (post-condition) would be after the occurrence of a cardiac event. The acquisition of the ECG data for three different states of the subject ensures that the models (the first training model 110 and the second training model 114) learn across different states of each patient.

The plurality of sets of ECGs (104-A-C) of the plurality of subjects, thus received by the quantifier 106 are converted into their corresponding time series signal 108-A. In one embodiment, the quantifier 106 is configured for extracting a first approximation of the time series signal 108-A from each of the sets of ECG (104-A-C) of the plurality of subjects. The analysis of electrocardiogram morphology provides the heart rate variability (HRV) of the subject. The heart rate variability consists of changes in the time intervals between consecutive heartbeats and are called inter beat intervals (IBIs). As a result, given the input data, in time domain, the quantifier 106 is configured for identifying the time domain features signal characteristics (HRV time-domain measures) such as: SDNN (Standard Deviation of Normal to Normal (heart rate)), SDRR, SDANN, SDNN Index, pNN50, HR Max−HR Min, RMSSD, HRV triangular index and TINN.

It is to be noted that, a signal prior to extracting the first approximation of the time series signal 108-A by the quantifier 106 may also be fed to a third training model for detecting a cardiac event. The details with respect to detection of the cardiac event from the ECG images (104-A-C) is described in detail in FIG. 4 below.

In one embodiment, the steps performed by the quantifier 106 for extracting the first approximation of the time series signal 108-A from the plurality of sets of ECGs (104-A-C) comprise peak detection, baseline extraction, block formation, and character identification. In one embodiment, methods known in art are implemented by the quantifier 106 for performing peak detection, baseline extraction, block formation and character identification on the images obtained by the sets of ECGs (104-A-C), for extracting the first approximation of the time series signal 108-A.

A manner in which the the quantifier 106 of FIG. 1 operates for peak detection from sets of ECG (104-A-C) is described in detail further below.

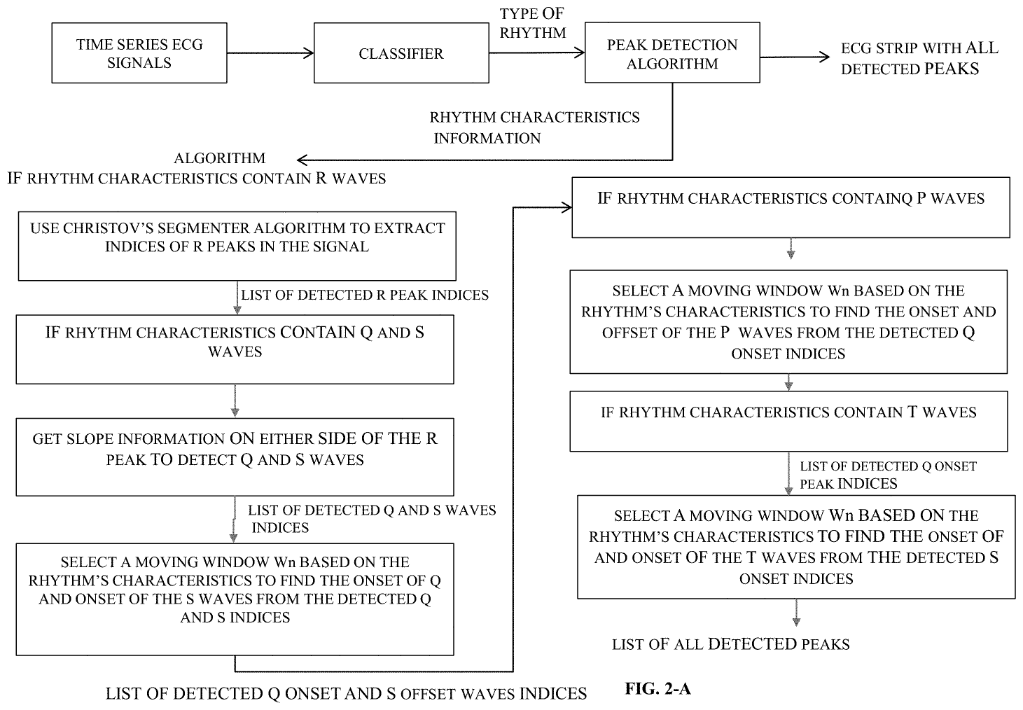

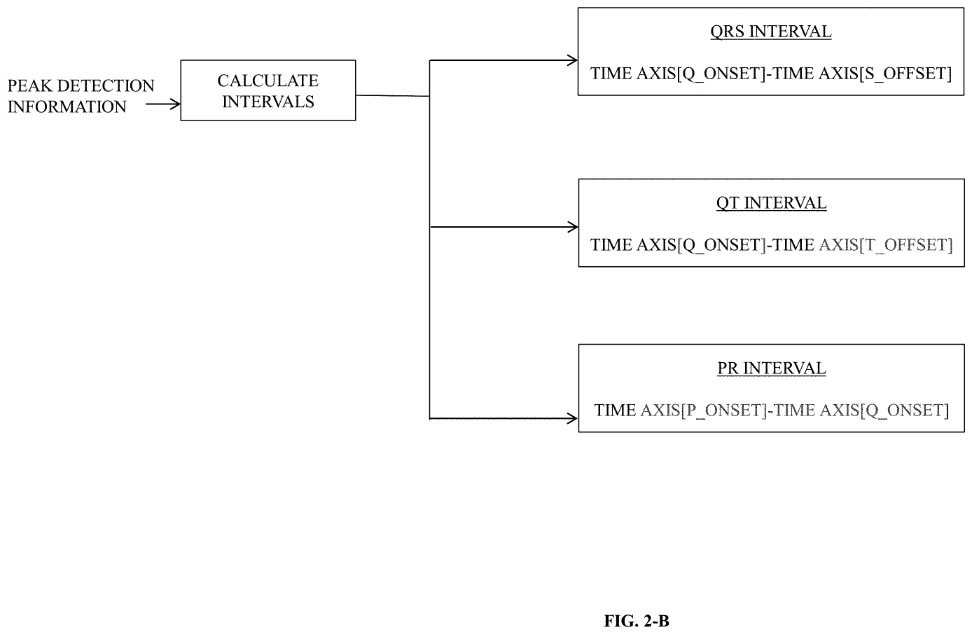

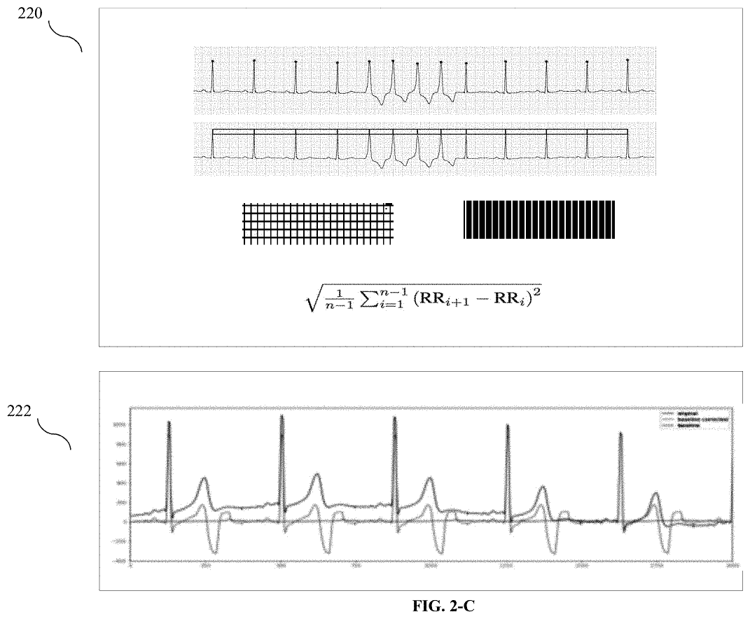

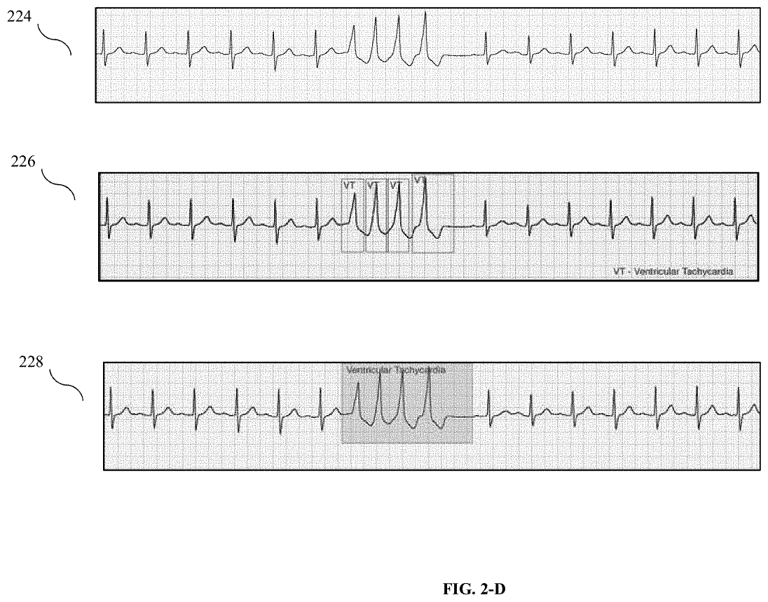

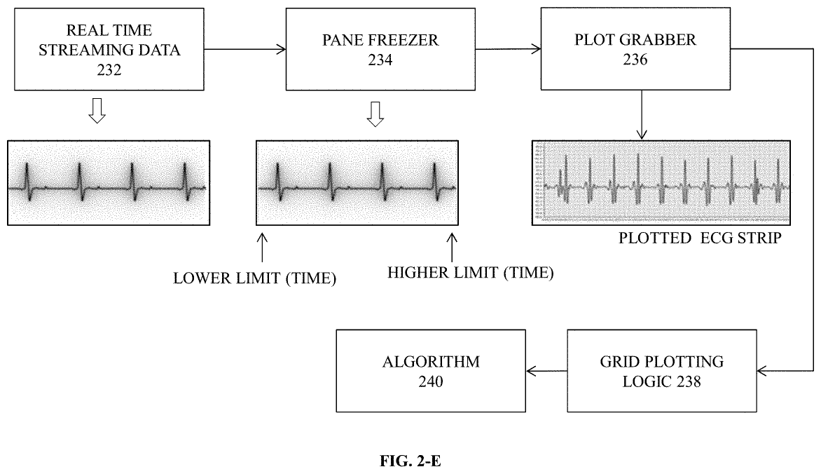

FIG. 2-A-B is an exemplary illustration of a peak detection algorithm implemented for extracting time series signals from each of the sets of electrocardiograms (104-A-C), in accordance with an embodiment of the present disclosure. In one example, an automatic peak detection algorithm (as shown in FIGS. 2-A and 2-B) is implemented and applied to the image data obtained from the plurality of sets of ECG (104-A-C). The process involved for peak detection from sets of ECGs (104-A-C) is described in below.

In one example, the coordinates (column and row number) of the pixels in the original input image (ECG 104-A-C), which is in two dimensions, where the pixel value is less than 80 are retrieved and stored in two separate lists X and Y. For R Peak Detection, a list named ‘peaks’ is created. The next step involves, looping through the list X and computing the difference between current point and the next point. If the difference is greater than 30 and that element is not existing in the list, coordinates are appended to a list. The exemplary illustration of the referenced steps is as shown in FIG. 2-C by reference numeral 220.

Eg: if (X[i]−X[i+1])>30:
if X[i] not in the list:
   peaks. add(X[i], Y[i])
The list named ‘peaks’ now comprises of the X and Y coordinates (column and row number) of all the R peaks in the original input image.

The quantifier 106 is configured for performing baseline extraction subsequent to the step of peak detection on the sets of ECGs (104-A-C) images. For baseline extraction, the dimensionality is reduced and ECG is extracted from ECG images (104-A-C). The coordinates of the pixels, where the pixel value is less than 80 in the input two-dimensional grayscale image (row and column number) are retrieved to ensure that pixels denoting the ECG grids are not considered for further pre-processing. The X and Y coordinates (column and row number of the array) obtained are stored in two separate lists. The original input image in two dimensions is thus reduced to two one dimensional lists. The step involved in baseline extractions includes reference wave generation. The X-coordinate list and the Y-coordinate list obtained in the previous step are used as input to create a one-dimensional array using a method shown by the following pseudo code:
ref_points[0]=X[0]
for i in range (l, length of X):
ref_points[i]=m+X[i]
   where m is the slope between points (Xi, Yi) and (Xi+1, Yi+1) given by: slope=(y2−y1)/(x2−x1)

Once the reference image is generated, baseline is extracted either using wavelet transformation and median technique as depicted in FIG. 2-C by reference numeral 222.

The quantifier 106 is configured for, subsequent to the step of baseline extraction, block formation and object detection. Input greyscale image is fed to an object detection model. The exemplary illustration of the Input greyscale image fed to an object detection model is as shown in FIG. 2-D by reference numeral 224.

The object detection model returns the following tuple and is as shown in FIG. 2-D by reference numeral 226.
X, Y, width and height of each of the ‘n’ bounding boxes
Class probability score for each bounding box
Sort the tuple obtained in step 2 by X
The total width is computed by:

total_width=Xbounding box n−Xbounding box 1
Set X_final as Xbounding box 1 and Y_final as Ybounding box 1
Set total_height as max(heightbounding box 1,heightbounding box 2, . . . heightbounding box n)
The bounding box is created for the following tuple:—is as shown in FIG. 2-D by reference numeral 228.
X_final,Y_final,total_width,total_height

With this method as described above and with every cardiac beat (R-R interval), it demonstrates the ability to identify the type of arrhythmia as well as helping to characterize the ECG signal by converting the image-based ECG data into time-series based data.

The quantifier 106 is configured for performing character identification subsequent to the step of block formation and object detection. The time series ECG signal 108-A thus obtained is further described in the chart below to detect ‘R’ peak. The ‘R’ peak found in time series data is compared with the ‘R’ peak obtained from ECG strip chart using 2D convolution to ascertain the correctness. Any noise associated with the time series signal 108-A is removed using wavelet transformation techniques. Once ‘R’ peaks are detected, the intervals are computed to conclude the ECG characterization using data transformation techniques.

The above paragraphs and FIGS. 2-A through 2-D depict, the exemplary steps performed by the quantifier 106 for extracting the first approximation of the time series signal 108-A, from each of the sets of ECG 104-A-C.

In one embodiment, the training dataset is created from the extracted time series signals 108-A and the raw time series signal 108-B (electrocardiograph signals) and stored in first training database 112 for training the first model 110. The first model 110 is trained for extracting the second approximation of the time series signal from the electrocardiogram of the subject under test. The second approximation represents an electrocardiograph signal that produced the electrocardiogram of the subject under test

In one embodiment, the first training model 110 is CNN 1D model. The Convolutional Neural Network (CNN) models are used for image classification, in which the model accepts a two-dimensional input representing an image, in a process called feature learning. A one-dimensional CNN model has a convolutional hidden layer that operates over a 1D sequence. This may be followed by a second convolutional layer in some cases, such as long input sequences, and then a pooling layer whose job it is to distil the output of the convolutional layer to the most salient elements.

Furthermore, a second training model 114 is trained on the training dataset, using machine learning, for extracting the cardiac marker 120 from the second approximation of the time series signal extracted from the electrocardiogram of the subject under test for calculating a probability (prediction 126) of occurrence of a cardiac event.

For training the second training model (ANN) 114, and to extract the cardiac markers 120, the key ECG characteristics obtained and extracted from the ECG across various patient states are then fed into second training model 114 along with several co-morbidities and features 116 about the patient from the Electronic Medical Records (EMR) 118. The second training model 114 may be Artificial Neural Network (ANN) model. To extract the accurate cardiac markers 120 each set of electrocardiograms is cross referenced with electronic medical records of each of the subjects from whom the electrocardiograms were recorded. Further, a probability of occurrence of a cardiac event is calculated based on the electronic medical records of the subject under test.

In one example, the training dataset is created in a similar way, with each of the dataset that is pre-occurrence of the cardiac event, at event, and post event of the cardiac event. This helps in generating a key feature, a cardiac marker 120 that is further fed into the second model 114 (ANN) along with several other features such as patient prior episodes, history, medications, laboratory tests, etc., from other sources of data. Heart Rate Variability (HRV), a commonly used parameter to measure the autonomous nervous system is computed across all sets of cardiac events to act as a feature to ultimately generate a probability that an arrhythmia can occur in the near future.

Thus, the cardiac marker 120, generated by the second training model 114 (ANN), can be used essentially for predicting a probability that the subject under test is developing an irregular heart rhythm. Thus the first training model 110 and the second training model 114 are trained to estimate at least one of a presence of a cardiac marker 120 indicating a prior occurrence of a cardiac event and calculating a probability of a future occurrence of a cardiac event from the electrocardiogram of the subject under test.

As described above, each set of electrocardiograms (104-A-C) is cross referenced with electronic medical records 118 of each of the subjects from whom the electrocardiograms were recorded for calculating the probability of occurrence of the cardiac event based on the electronic medical records 118 of the subject under test. The probability of occurrence of cardiac events are known to be influenced by risk factors such as hypertension, diabetes, life style, etc. The probability of occurrence of a cardiac event predicted based on, apart from the cardiac markers, that indicate the probability may be made more accurate and dependable by using this additional information and extracting a correlation between the risk factors and the historical occurrence of the cardiac events in the electronic medical records 118 and ECGs in the dataset.

The key ECG characteristics such as R-R interval, QRS, ST segment, etc., are obtained by comparing the ECG features from the extracted time series signal 108-A with the raw time series signal 108-B that is fed directly into first training model 110 (CNN 1D). This ensures that the time series signal 108-A extracted from the ECG images 104-A-C is calibrated as close as possible to the raw time series signal 108-B fed directly into first training model 110 (CNN 1D). Thus, the calibration also enables cross-learning technique of 2D images obtained from sets of ECG (104-A-C) with the 1D time series data (raw time series signal 108-B) for error detection and re-training.

In one embodiment, cross-learning technique of 2D images with 1D time-series data for detection to create a hybrid training dataset is achieved. Given the classification of 1D time-series data is proven and robust, the 1D classification is treated as the gold standard which in turn is used to validate the training dataset on trained from 2D images. Given this, a subset of the training dataset from the 2D images are used as validation set with the training dataset of the 1D time-series data. Any mis-classification is computed and fed back into the system 100 for re-training and classification.

Furthermore, in another embodiment of the present disclosure, a cardiac event, of the subject under test, may also be predicted and detected, by performing image analysis, on a live electrocardiogram computed from near real time electrocardiograph signals of the subject under test. The received near real time electrocardiograph signals are of the subject under test, undergoing at least one of remote monitoring, continuous monitoring, and ambulatory monitoring. The electrocardiograms are computed from the received electrocardiograph signals of the subject under test by sequentially performing pane freezing, plot grabbing and grid-plotting.

Referring to FIG. 1, the raw time series signal 108-B obtained in real-time can also be fed to the visualizer 124 which aids in converting the raw time series signal 108-B to corresponding ECG images (104-D-F) of the plurality of subjects.

In one exemplary illustration as shown in FIG. 2-E, a Pane Freezer 234 and a Plot Grabber 236 are implemented to convert the real time streaming data (electrocardiograph signal 108-B) 232 to the corresponding ECG (104-D-F) strip chart. The ECG (104-D-F) image strip is again fed back into the quantifier 106 to be able to detect or predict any cardiac event of the subject under test. For example, in a situation where there is continuous monitoring of ECG (such as in critical care, operation theater, emergency room, etc.), the real-time streaming data captured can be computed at any instant to detect and predict arrhythmia conditions using the method 240 and by implementing the method comprising the steps as:
(i) Set the big box and the small box intervals on the x-axis, that is, (0.2 seconds for the big boxes and 0.04 seconds for the small boxes) by dividing the length of the ECG strip (in seconds);
(ii) Set the Y major and the minor intervals that is, (0.5 mV for big and 0.1 mV for small boxes) depending on the maximum and the minimum value of the amplitude of the signal by dividing them by 0.5 mV and 5 mV respectively;
(iii) Plot the grid, based on markings using grid plotting logic 238; and
(iv) Calculate the x-axis/time axis of the signal using the sampling frequency information and plot the signal with its respective x-axis value on the plotted grid.

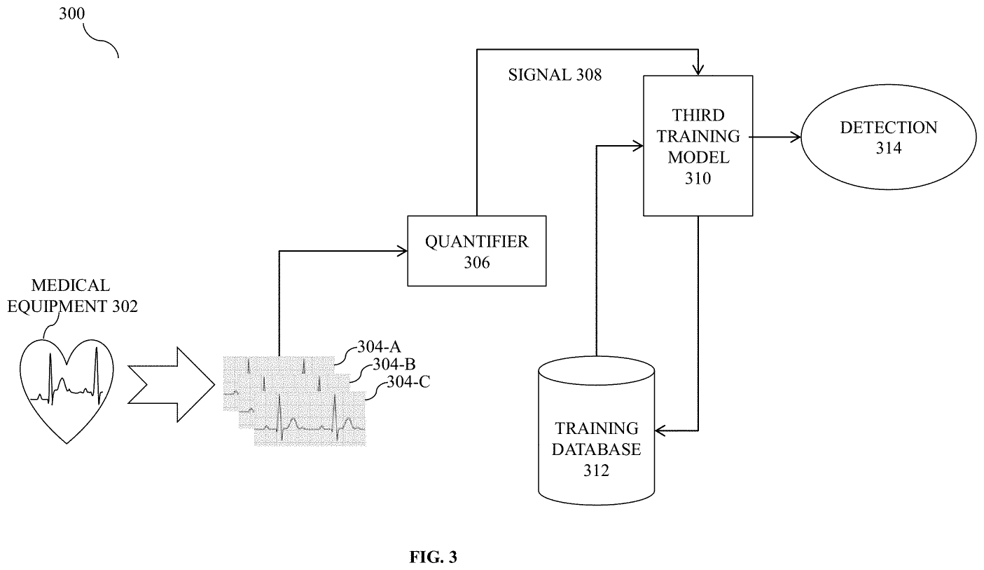

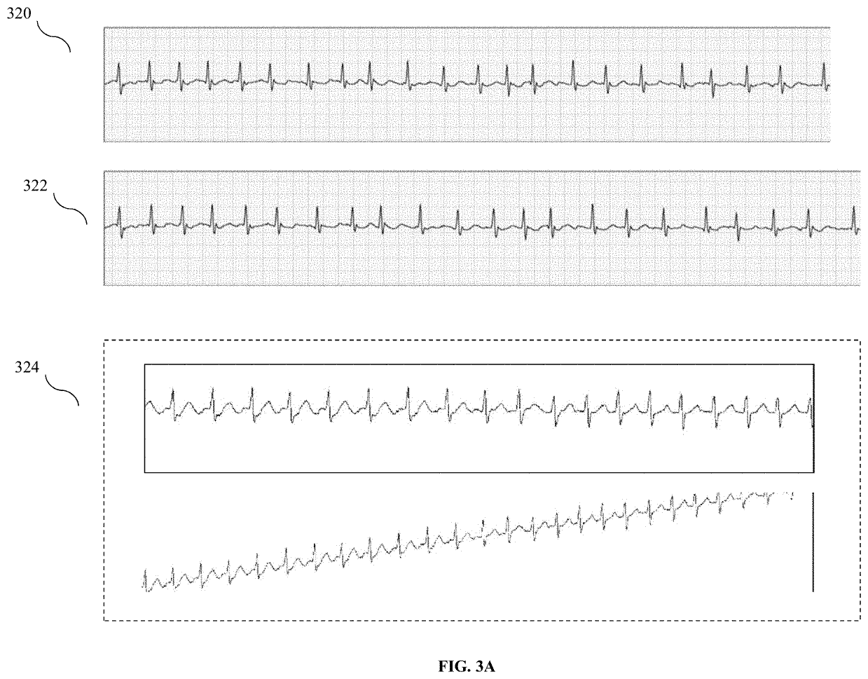

As described above, a signal prior to extracting the first approximation of time series signal 108-A from the quantifier 106 may also be fed to the third training model for detecting a cardiac event. The details with respect to detection of the cardiac event from the ECG images (104-A-C) is described in detail in FIG. 3 below.

FIG. 3 illustrates an exemplary environment for detecting the previous occurrence of cardiac event from an electrocardiogram of the subject under test, in accordance with an embodiment of the present disclosure. In particular, FIG. 3 illustrates, a medical equipment such as electrocardiograph 302, a plurality of sets of electrocardiograms (ECG 304-A-C) of the plurality of subjects, a quantifier 306, a signal 308 extracted from the quantifier 306, a third training model 310, a third training database 312, and a output signal 314 for detection of the cardiac event. Furthermore, the third training model 310 is trained using machine learning, for detecting the cardiac event using the two-dimensional electrocardiogram image and is described in detail further below.

The third training model 310 is trained using input data such as images obtained from the sets of ECGs (304-A-C) of the subjects. In one example, the ECG (304-A-C) illustrates the plot showing the electrical activity of a heart of the plurality of subjects on a strip chart.

Referring to FIG. 3, the plurality of sets of ECGs (304-A-C) of the plurality of subjects is received as the input data to the quantifier 306. Each set of ECGs of the subject comprises at least one ECG (304-A) obtained prior to the occurrence of a cardiac event, one ECG obtained during the occurrence of the cardiac event (304-B), and one ECG obtained after the occurrence of the cardiac event (304-C). The input data is analyzed by the quantifier 306 for detection of the cardiac event (arrhythmias). The analysis for detection of cardiac event by above method includes the steps of cropping and pre-processing of the image data acquired data from the sets of ECG (304-A-C) followed by sub-steps of hue saturation correction and data augmentation.

The analysis for detection of cardiac event is described in detail using an illustration as shown in FIG. 3-A. In one example, the detection may happen in three phases. The first phase is cropping and pre-preprocessing to ensure input data is consistent. The input data in this case is an ECG strip chart in any of the image formats into a {x by y} pixel that is pre-defined or configured to any size that the model can be trained. An illustration of a 1000 by 100 pixel format on which the model is being trained is shown in FIG. 3-A by reference numeral 320.

The second phase is Hue and Saturation correction—In order that the third training model can understand region of interest, hue and saturation corrections are applied on input data as shown in FIG. 3-A by reference numeral 322 to extract details.

The next step (third phase) to detect arrhythmia using 2D ECG strip chart images is data augmentation that involves rescaling, adjusting rotation range up to 30 degrees, width and height shift range of 0.2 and shear/zoom range to be approximately 0.2 as shown in FIG. 3-A by reference numeral 324.

The data obtained from image analysis of the ECG (304-A-C) is then fed to the third training model 310. In one example, the third training model 310 may be a two-dimensional convolution neural network (CNN 2 D). The data acquired at this step (from CNN2D) is stored in training database 312 for learning and future analysis and for detecting (314) the cardiac event. Thus, a standard two-dimensional convolution neural network is implemented to classify the image based on the above pre-processing techniques.

Hence, the embodiments disclosed in the present disclosure may be implemented for predicting and detecting predicting an occurrence of a cardiac event, from any form of input data such as ECG data either in the time series or that of a strip chart. The embodiments of the present disclosure intelligently transform data from one domain to the other and helps detect and predict irregular heart rhythms of patients, while constantly ensuring image data sets are as close as possible to real-time series data.

Furthermore, the data transformation and cross-learning techniques implemented by the embodiments disclosed in the present disclosure provides with a very high sample data and thus enables significantly higher accuracy, precision and recall scores in detecting and predicting arrhythmias compared to traditional methodologies. Moreover, a cardiac marker predicted herein helps quantify the risk factor for a patient that can be further used to manage the subject proactively. Since, the embodiments of the present disclosure can be implemented across time series signals and images, in the case where a patient in ambulatory, or visiting a diagnostic center, a mere photograph using a camera of the ECG strip chart can be used to classify and predict arrhythmias.

FIG. 4 is a flow chart illustrating a method 400 for training one or more models, using machine learning, for predicting a probability of occurrence of a cardiac event from an electrocardiogram of a subject under test, in accordance with an embodiment of the present disclosure. FIG. 4 may be described from the perspective of a processor that is configured for executing computer readable instructions stored in a memory to carry out the functions of the modules (described below and not shown in the figures) of the system 100. In particular, the steps as described in FIG. 4 may be executed for training one or more models, using machine learning, for predicting a probability of occurrence of a cardiac event from an electrocardiogram of a subject under test. Each step is described in detail below.

At step 402, a plurality of sets of electrocardiograms of a plurality of subjects is obtained. Each set of electrocardiograms for each subject includes at least one electrocardiogram obtained prior to an occurrence of a cardiac event and at least one of an electrocardiogram obtained during an occurrence of the cardiac event and an electrocardiogram obtained after an occurrence of a cardiac event. The plurality of sets of ECGs of the plurality of subjects is received as input data. The ECG data which includes the plurality of sets of ECGs is collected and received across different states (state A, state B, and state C) of the plurality of subjects over a period of time as shown in FIG. 1-A-B. It is to be noted that the word, “a” cardiac event is used multiple times, for a subject under test to signify that there is a possibility of occurrence of a number of cardiac events for the same subject under test.

At step 404, a first approximation of a time series signal is extracted from each of the electrocardiograms from the plurality of sets of electrocardiograms which are obtained at step 402. In one embodiment, the quantifier is configured for extracting a first approximation of the time series signal from each of the sets of ECGs of the plurality of subjects.

At step 406, each of an electrocardiograph signal that produced each of the electrocardiograms is obtained. The electrocardiograph signal is a raw time series signal. In one example, the electrocardiograph signal is the detected signal (raw time series signal) obtained from an electrocardiograph. The electrocardiograph is a medical equipment, comprising of electrodes for detecting the electrical activity of the heart and the detected signal is used to produce the ECG. In the same example, the electrocardiograph signal is the raw time series signal obtained in the real time and that produced each of the electrocardiograms.

At step 408, a training dataset is created from the extracted time series signals and the electrocardiograph signal for training a first model. The created training data set is used for training the first model for extracting a second approximation of a time series signal from the electrocardiogram of the subject under test. The second approximation represents an electrocardiograph signal that produced the electrocardiogram of the subject under test

At step 410, a second training model is trained on the training dataset for extracting a cardiac marker from the second approximation of the time series signal extracted from the electrocardiogram of the subject under test. For training the second training model, and to extract the cardiac markers, the key ECG characteristics obtained and extracted from the ECG across various patient states are fed into second training model along with several co-morbidities and features about the patient from the Electronic Medical Records (EMR). The second training model may be Artificial Neural Network (ANN) model.

At step 412, a probability of occurrence of a cardiac event is calculated. In one example, to extract the accurate cardiac markers each set of electrocardiograms is cross referenced with electronic medical records of each of the subjects from whom the electrocardiograms were recorded. Further, a probability of occurrence of a cardiac event is calculated based on the electronic medical records of the subject under test.

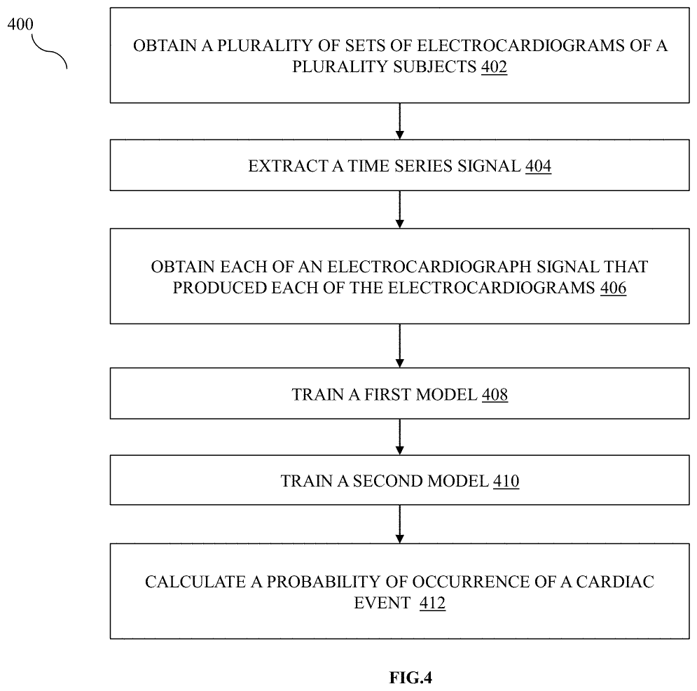

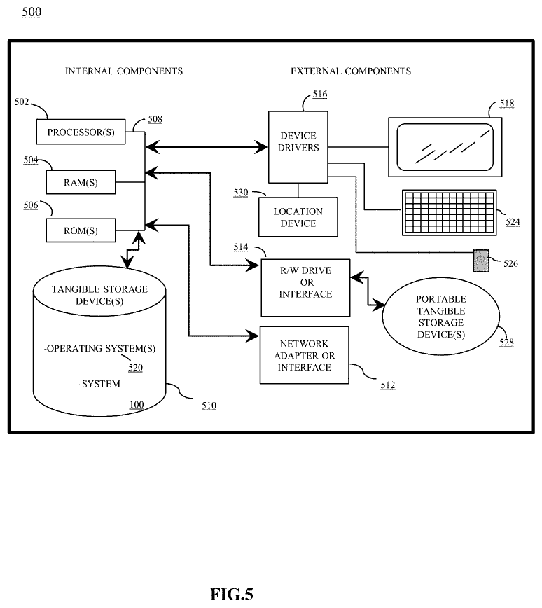

FIG. 5 is a block diagram 500 for of a computing device utilized for implementing the system 100 of FIG. 1 implemented according to an embodiment of the present disclosure. The modules of the system 100 described herein are implemented in computing devices. The computing device 500 comprises one or more processor 502, one or more computer-readable RAMs 504 and one or more computer-readable ROMs 506 on one or more buses 508.

Further, the computing device 500 includes a tangible storage device 510 that may be used to execute operating systems 520 and modules existing in the system 100. The various modules of the system 100 can be stored in tangible storage device 510. Both, the operating system and the modules existing in the system 100 are executed by processor 502 via one or more RAMs 504 (which typically include cache memory).

Examples of storage devices 510 include semiconductor storage devices such as ROM 506, EPROM, flash memory, or any other computer-readable tangible storage device 510 that can store a computer program and digital information. Computing device also includes R/W drive or interface 514 to read from and write to one or more portable computer-readable tangible storage devices 528 such as a CD-ROM, DVD, and memory stick or semiconductor storage device. Further, network adapters or interfaces 512 such as a TCP/IP adapter cards, wireless WI-FI interface cards, or 3G or 4G wireless interface cards or other wired or wireless communication links are also included in computing device 500. In one embodiment, the modules existing in the system 100 can be downloaded from an external computer via a network (for example, the Internet, a local area network or other, wide area network) and network adapter or interface 512. Computing device 500 further includes device drivers 516 to interface with input and output devices. The input and output devices can include a computer display monitor 518, a keyboard 524, a keypad, a touch screen, a computer mouse 526, and/or some other suitable input device.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

We claim:

1. A system for predicating and detecting a cardiac event from an electrocardiogram of a subject under test, the system comprising:
a processor with a memory, the memory storing a plurality of modules configured for at least one of detecting and predicting an occurrence of a cardiac event from the electrocardiogram of the subject under test; wherein the plurality of modules are characterized by:
a first module having been trained using machine learning and configured for:
obtaining a plurality of sets of electrocardiograms of a plurality of subjects, wherein each set of electrocardiograms for each subject comprises at least one electrocardiogram obtained prior to an occurrence of a cardiac event and at least one of an electrocardiogram obtained during an occurrence of the cardiac event and an electrocardiogram obtained after an occurrence of a cardiac event;
extracting a first approximation of a time series signal from each of the electrocardiograms;
obtaining each of an electrocardiograph signal that produced each of the electrocardiograms; wherein the electrocardiograph signal is a raw time series signal;
creating a training dataset from the extracted time series signals and the electrocardiograph signals and training a first model on the training dataset for extracting a second approximation of a time series signal from an electrocardiogram of the subject under test, wherein the second approximation represents an electrocardiograph signal that produced the electrocardiogram of the subject under test; and
a second module comprising a second model, having been trained using machine learning, on the training dataset for extracting a cardiac marker from the second approximation of the time series signal extracted from the electrocardiogram of the subject under test and calculating a probability of occurrence of a cardiac event.

2. The system as claimed in claim 1, wherein each set of electrocardiograms is cross referenced with electronic medical records of each of the subjects from whom the electrocardiograms were recorded and calculating the probability of occurrence of the cardiac event based on the electronic medical records of the subject under test.

3. The system as claimed in claim 1, comprising a third module comprising a third model having been trained using machine learning for detecting the previous occurrence of cardiac event from the electrocardiogram of the subject under test, by creating a third training dataset by performing image analysis on each of the obtained electrocardiograms.

4. The system as claimed in claim 1, comprising predicting and detecting a cardiac event, by performing image analysis, on a live electrocardiogram computed from near real time electrocardiograph signals received of the subject under test.

5. The system as claimed in claim 4, wherein the received near real time electrocardiograph signals are from the subject under test undergoing at least one of remote monitoring, continuous monitoring and ambulatory monitoring.

6. The system as claimed in claim 1, comprising computing electrocardiograms from the electrocardiograph signal received, of the subject under test by sequentially performing pane freezing, plot grabbing and grid-plotting.

7. A method for training one or more models, using machine learning, for predicting a probability of occurrence of cardiac event from an electrocardiogram of a subject under test, the method comprising:
obtaining a plurality of sets of electrocardiograms of a plurality of subjects, wherein each set of electrocardiograms for each subject comprises at least one electrocardiogram obtained prior to an occurrence of a cardiac event and at least one of an electrocardiogram obtained during an occurrence of the cardiac event and an electrocardiogram obtained after an occurrence of a cardiac event;
extracting a first approximation of a time series signal from each of the electrocardiograms;
obtaining each of an electrocardiograph signal that produced each of the electrocardiograms; wherein the electrocardiograph signal is a raw time series signal;
creating a training dataset from the extracted time series signals and the electrocardiograph signals and training a first model on the training dataset for extracting a second approximation of a time series signal from the electrocardiogram of the subject under test; wherein the second approximation represents an electrocardiograph signal that produced the electrocardiogram of the subject under test;
training a second model on the training dataset for extracting a cardiac marker from the second approximation of the time series signal extracted from the electrocardiogram of the subject under test; and
and calculating a probability of occurrence of a cardiac event.

8. The method as claimed in claim 7, wherein each set of electrocardiograms is cross referenced with electronic medical records of each of the subjects from whom the electrocardiograms were recorded and calculating the probability of occurrence of the cardiac event based on the electronic medical records of the subject under test.

9. The method as claimed in claim 7, comprising creating a third training dataset by performing image analysis on each of the obtained electrocardiograms for training a third model for detecting the previous occurrence of cardiac event from the electrocardiogram of the subject under test.

10. The method as claimed in claim 7, comprising predicting and detecting a cardiac event, by performing image analysis, on a live electrocardiogram computed from near real time electrocardiograph signals received of the subject under test.

11. The method as claimed in claim 7, wherein the received near real time electrocardiograph signals are from the subject under test undergoing at least one of remote monitoring, continuous monitoring and ambulatory monitoring.

12. The method as claimed in claim 11, comprising computing electrocardiograms from the electrocardiograph signals received, of the subject under test by sequentially performing pane freezing, plot grabbing and grid-plotting.
